(19) United States
(12) Patent Application Publication
(10) Pub. No.: US 2025/0367904 A1
(43) Pub. Date: Dec. 4, 2025

(54) LAMINATED FIBER SHEET AND METHOD OF USING THE SAME

(71) Applicant: Kao Corporation, Tokyo (JP)

(72) Inventors: Yoshihiko KONDO, Tochigi (JP); Takehiko UEMATSU, Tochigi (JP); Yuta ABE, Tochigi (JP); Mahoko ONO, Tochigi (JP); Takehiko TOJO, Tochigi (JP)

(73) Assignee: Kao Corporation, Tokyo (JP)

(21) Appl. No.: 19/303,427

(22) Filed: Aug. 19, 2025

Related U.S. Application Data
(63) Continuation of application No. PCT/JP2024/005524, filed on Feb. 16, 2024.

(30) Foreign Application Priority Data
Feb. 22, 2023 (JP) 2023-026742
Feb. 22, 2023 (JP) 2023-026743

(51) Int. Cl. B32B 5/26; B32B 5/02

(52) U.S. Cl. CPC B32B 5/267; B32B 5/022; B32B 2250/02; B32B 2250/20; B32B 2262/0253; B32B 2307/714; B32B 2307/718; B32B 2307/726; B32B 2307/737

(57) ABSTRACT

A method regarding a laminated fiber sheet, wherein the laminated fiber sheet can be moistened with a liquid substance, and the laminated fiber sheet can include an outer sheet and an inner sheet which is laminated on one face of the outer sheet, wherein, in the laminated fiber sheet, the inner sheet and the outer sheet are insoluble to a liquid substance, the inner sheet has a median fiber diameter of 0.3 μm or more and 5 μm or less, the outer sheet has a larger median fiber diameter than the inner sheet, and the inner sheet and the outer sheet are integrated. The method may include a step of applying the laminated fiber sheet to an object with the inner sheet facing toward the object; and a step of applying the liquid substance.

### CROSS-REFERENCE TO RELATED APPLICATIONS

This application is a Continuation of PCT International Application No. PCT/JP2024/005524 filed on Feb. 16, 2024, which claims priority under 35 U.S.C. § 119 (a) to Japanese Patent Application No. 2023-026742 filed in Japan on Feb. 22, 2023, and Japanese Patent Application No. 2023-026743 filed in Japan on Feb. 22, 2023. Each of the above applications is hereby expressly incorporated by reference, in its entirety, into the present application.

### SUMMARY

According to an aspect, a method regarding a laminated fiber sheet can be implemented. The laminated fiber sheet can be moistened with a liquid substance.

The laminated fiber sheet can be provided and can include an outer sheet and an inner sheet which is laminated on one face of the outer sheet.

The inner sheet and the outer sheet can be insoluble to the liquid substance.

The inner sheet can have a median fiber diameter of 0.3 μm or more and 5 μm or less.

The outer sheet can have a larger median fiber diameter than the inner sheet.

The inner sheet and the outer sheet can be integrated.

According to another aspect, a laminated fiber sheet can include an outer sheet and an inner sheet which is laminated on one face of the outer sheet.

The laminated fiber sheet can have a structure of the inner sheet and the outer sheet described above in the preceding paragraph(s).

### BRIEF DESCRIPTION OF DRAWINGS

FIG. 1 is a cross-sectional view schematically illustrating one or more embodiments of a laminated fiber sheet according to the present disclosure.

FIG. 2(A) is a photograph substituting for a drawing illustrating a state where an inner sheet alone is pinched by fingers.

FIG. 2(B) is a photograph substituting for a drawing illustrating a state where the laminated fiber sheet of the present invention is pinched by fingers.

FIG. 3 is an explanatory view schematically illustrating a fusion bonding point at an intersection point of ultra-fine fibers of the inner sheet.

FIG. 4 is a photograph substituting for a drawing illustrating an exemplary observation image used to measure a proportion of the number of fusion bonding points occupied in the number of fiber intersection points.

FIG. 5 is a schematic diagram illustrating an example in which the fusion bonding points are included at the intersection points of both fibers at an interface between the inner sheet and the outer sheet.

FIG. 5(A) illustrates a state before extension, and FIG. 5(B) illustrates an extended state.

FIG. 6 is an explanatory view schematically illustrating a state where one example of the laminated fiber sheet of the present invention is applied to an object.

FIG. 7(A) is a photograph substituting for a drawing illustrating a transfer result of a liquid substance from the outer sheet to filter paper in Example 1.

FIG. 7(B) is a photograph substituting for a drawing illustrating a transfer result of the liquid substance from the inner sheet to filter paper.

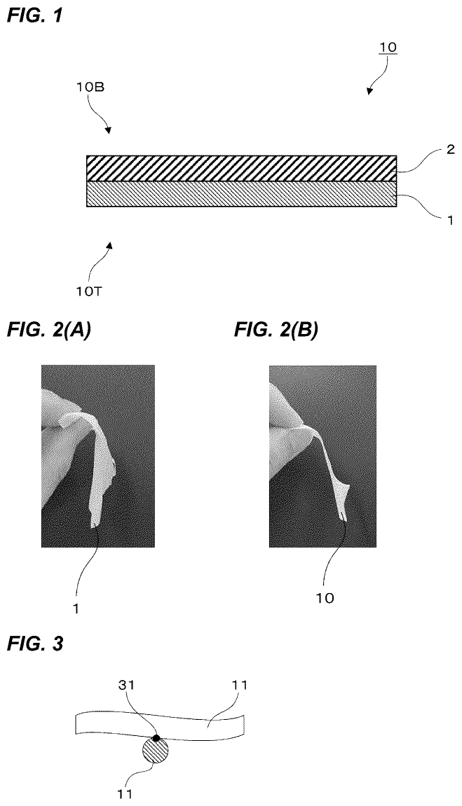

### DESCRIPTION OF EMBODIMENTS

The present disclosure and one or more embodiments thereof can relate to or include or involve a laminated fiber sheet and/or method thereof that can create a persistent wet state of a liquid substance simultaneously while enabling stuffiness prevention and stickiness suppression on an outer side thereof.

Other and further one or more objects, features and/or advantages will appear more fully from the following description, appropriately referring to the accompanying drawings.

JP-A-2008-179629 (“JP-A” means unexamined published Japanese patent application) describes a sheet containing ultra-fine fibers. However, conventionally, such an ultra-fine fiber sheet has a high permeability of a liquid substance, and therefore, when it is used alone, not only a surface opposed to the object, but also an outer surface on an opposite side of the ultra-fine fiber sheet easily enters a wet state. Therefore, the ultra-fine fiber sheet that retains a liquid substance becomes sticky on the outer side in a state where the ultra-fine fiber sheet is affixed to the object, and the liquid substance is transferred to surrounding objects in some cases.

JP-A-2017-137585 describes a material having a liquid proofing property, such as plastics.

A laminated fiber sheet of one or more embodiments the present disclosure and/or method thereof can create a persistent wet state of a liquid substance simultaneously while enabling stuffiness prevention and stickiness suppression on the outer side.

Where a numerical limit or range is stated in the specification, the endpoints are included. Also, all values and subranges within a numerical limit or range are specifically included as if explicitly written out.

Singular terms used in the specification carry the meaning of “one or more” and/or “at least one.”

Numerous modifications and variations of one or more embodiments of the present disclosure are possible in light of the above and below shown disclosures. It is therefore to be understood that, within the scope of the present disclosure, including the claims, one or more embodiments may be practiced otherwise than as specifically described herein.

All patents and other references mentioned above and below are incorporated in full herein by this reference, the same as if set forth at length.

The following describes a preferred embodiment of the laminated fiber sheet of the present disclosure by referring to the drawings.

A laminated fiber sheet 10 can include an outer sheet 2 and an inner sheet 1 which can be laminated on one face of the outer sheet 2. The inner sheet 1 is placed on one face side 10T of front and back faces of the laminated fiber sheet 10, and the outer sheet 2 is placed on the other face side 10B on the opposite side to the one face side 10T.

The laminated fiber sheet 10 is typically applied to an object with the inner sheet 1 facing toward the object described below in a method of using the laminated fiber sheet 10. Application to an object can mean that the laminated fiber sheet 10 is brought into contact with a surface of the object. The laminated fiber sheet 10 is preferably affixed to the object by an action of moistening of the laminated fiber sheet 10.

The laminated fiber sheet 10 may include another fiber layer in addition to the inner sheet 1 and the outer sheet 2. In this case, from the perspective of allowing characteristics of the inner sheet 1 described below to significantly work and not impairing a cooperative action of the inner sheet 1 and the outer sheet 2, for instance, the other fiber layer is preferably located on the other face side 10B of the outer sheet 2. That is, the inner sheet 1 and the outer sheet 2 can be adjacent in one or more embodiments of the present disclosure.

The laminated fiber sheet 10 typically may be put into a moistened state by containing a liquid substance in a using method, such as described herein. More specifically, the inner sheet 1 (in particular, the surface of the inner sheet 1 opposed to the object) is put into a moistened state with the liquid substance. The “moistened state” or “wet state” can mean a state where the laminated fiber sheet 10 is moistened with the liquid substance, that is, a state where the liquid substance exists. The method of using the laminated fiber sheet 10 and operations of the laminated fiber sheet 10 to the liquid substance in the using method are described later.

In the laminated fiber sheet 10, the amount of application of the liquid substance to enter a wet state is preferably 0.2 mg/cm² or more, more preferably 0.3 mg/cm² or more, further preferably 0.4 mg/cm² or more, and preferably 10 mg/cm² or less, more preferably 7 mg/cm² or less, further preferably 5 mg/cm² or less, and even further preferably 4 mg/cm² or less.

The “liquid substance” can mean a substance in liquid form at 20° C. In addition, the “liquid substance” in the present disclosure can refer to a substance that moistens a laminated fiber sheet. Examples of the liquid substance can include, for example, water, liquids, such as aqueous solutions and aqueous dispersions, gel-like substances increased in viscosity with thickeners, oils that are liquid or solid at 20° C., oil solutions containing 10 mass % or more of the oils, and emulsions (O/W emulsions and W/O emulsions) containing the oils and surfactants, such as nonionic surfactants, and the like.

The liquid substance preferably contains polyol that is liquid at 20° C.

This can make it hard for deactivation in a transparent state caused by evaporation to occur in the liquid substance. As the polyol, for example, one kind or two or more kinds selected from ethylene glycol, propylene glycol, 1,3-butanediol, dipropylene glycol, polyethylene glycol having a mass average molecular weight of 2 000 or less, glycerin, and diglycerin can be contained.

The liquid substance may contain an oil that is liquid at 20° C. As the oil, one kind or two or more kinds selected from hydrocarbon oils, esters made from a straight-chain or branched-chain fatty acid and a straight-chain or branched-chain alcohol or polyhydric alcohol, ester oils, and silicone oils can be contained.

As the hydrocarbon oils, one kind or two or more kinds selected from liquid paraffin, squalane, squalene, n-octane, n-heptane, cyclohexane, light isoparaffin, and liquid isoparaffin can be contained.

As the esters made from a straight-chain or branched-chain fatty acid and a straight-chain or branched-chain alcohol or polyhydric alcohol, one kind or two or more kinds selected from octyldodecyl myristate, myristyl myristate, isocetyl stearate, isocetyl isostearate, cetearyl isononanoate, diisobutyl adipate, di(2-ethylhexyl) sebacate, isopropyl myristate, isopropyl palmitate, diisostearyl malate, neopentyl glycol dicaprate, and alkyl benzoate (having 12 to 15 carbon atoms) can be contained.

As the ester oils, one kind or two or more kinds selected from triglycerol fatty acid esters (triglycerides) can be contained.

As the triglycerol fatty acid esters, caprylic/capric acid triglyceride and the like can be contained.

As the silicone oils, one kind or two or more kinds selected from dimethylpolysiloxane, dimethylcyclopolysiloxane, methylphenyl polysiloxane, methylhydrogenpolysiloxane, and higher alcohol modified organopolysiloxane can be contained.

The liquid substance may contain an oil that is solid at 20° C. As the oil, one kind or two or more kinds selected from vaseline, cetanol, stearyl alcohol, ceramide, and the like can be contained.

Such a liquid substance is preferably an agent that does not contain medicines and is used for cosmetic or aesthetic purposes.

As the “objects” to which the laminated fiber sheet 10 may be applied, various kinds of objects may be included. Examples can include human skin (flesh), and further, without being limited by this, the objects may be human nails, teeth, gums and hair; non-human mammalian skin (hide), nails, teeth and gums; plant surfaces, such as branches and leaves; or the like.

The using method in which the laminated fiber sheet 10 is moistened with the liquid substance and applied to the object may have or serve a purpose, among one or more purposes, for example, to enhance the surface condition of the object cosmetically and aesthetically. For example, when the object is human skin, the using method is preferably used for any purpose of making the skin beautiful in appearance, including enhancing a barrier function and a moisture-retaining function of the skin, brightening the skin, hiding wrinkles and macules, and wearing makeup. Optionally, the using method according to one or more embodiments of the present disclosure may not include methods in which doctors or persons receiving the instruction of doctors perform surgery, treatment, or diagnosis on humans.

The inner sheet 1 and the outer sheet 2, that the laminated fiber sheet 10 can have, are preferably insoluble to the liquid substance.

Being insoluble to the liquid substance can mean that a change in the reduction of fiber diameter caused by dissolution is not observed. As but one example, it can mean that or include a 50 mm square laminated fiber sheet is dipped in the liquid substance corresponding to 10 times the mass of the laminated fiber sheet under an environment of 1 atmospheric pressure and 23° C., and after a lapse of 24 hours, a median fiber diameter of each layer after the dipping is equal to or greater than each median fiber diameter before the dipping. A measuring method is based on (Method of Measuring Median Fiber Diameter) described below.

The above measuring method may be applicable, regardless of whichever property the liquid substance has, as long as the liquid substance moistens the laminated fiber sheet, for instance.

For example, when the liquid substance is water, the measurement may be performed using deionized water.

The inner sheet 1 preferably contains fibers having a median fiber diameter (P1) of 0.3 μm or more and 5 μm or less (in other words, nanofibers). The fiber is also called ultra-fine fiber or nanofiber and can be formed by, for example, an electrospinning method. In the inner sheet 1, fineness of the fibers can suppress unevenness of a fiber unit according to one or more embodiments of the present disclosure more than fiber layers of conventional nonwoven fabrics, can increase a contact area with an object surface (for example, a skin surface) and/or can increase adhesion to the object surface.

The outer sheet 2 preferably has a larger median fiber diameter than the inner sheet 1. Examples of the outer sheet 2 can include, for example, non-woven fabrics obtained by a spunbonding method (hereinafter referred to as spunbonded nonwoven fabrics).

In addition, the inner sheet 1 and the outer sheet 2 are preferably integrated according to one or more embodiments of the present disclosure. Specifically, the inner sheet 1 and the outer sheet 2 are preferably adjacent and integrated by fusion bonding of the fibers constituting the inner sheet 1 and the fibers constituting the outer sheet 2.

These can cause a capillary force from the outer sheet 2 toward the inner sheet 1 to act (e.g., extremely strongly) in a thickness direction of the laminated fiber sheet 10. In addition, the inner sheet 1 can be regarded as having a high liquid retention capacity because the specific surface area of the ultra-fine fibers is extremely large. And the inner sheet 1 can be regarded as having excellent performance of preventing liquid from returning to the outer sheet 2, which is the fiber layer layered on the inner sheet.

### Method of Measuring Median Fiber Diameter

(1) The laminated fiber sheet 10 is delaminated between fiber layers to extract the fiber layers to be measured. The fiber layer is cut into 10 mm×10 mm. This is preliminary affixed to a sample stage for scanning electron microscope (manufactured by Okenshoji Co., Ltd.) via conductive carbon double-sided tape (manufactured by Okenshoji Co., Ltd.).
(2) The sample stage to which the fiber layer is affixed is provided to a sputtering device (Ion Sputter E-1030, manufactured by Hitachi High-Tech Corporation). Pressure is reduced to 6 Pa under argon gas atmosphere and platinum-palladium (Pt—Pd) evaporation is performed. A distance between a fiber layer mounting surface and the Pt-Pd electrode is 30 mm, an evaporation time is 80 seconds, and a current value at the time of the evaporation is 30 mA.
(3) The sample stage is provided to a scanning electron microscope (SEM) (S-4300SE/N, manufactured by Hitachi High-Tech Corporation) to obtain observation images in high-definition mode (accelerating voltage: 5 kV, workpiece distance: 10 mm, observation magnification: 500 times or 1 000 times). The observation images of the same sample are obtained from a total of 15 locations by changing an observation location.
(4) The fiber diameter of the fiber layer is measured using an image analysis software (WinROOF2015, manufactured by MITANI CORPORATION) from the observation images obtained in the above (3). From the measured values of a total of 600 fiber diameters, a number average diameter, a number 10% diameter (D10) (from a small diameter side), a number 50% diameter (median diameter), and a number 90% diameter (D90) are calculated. Of these, the median diameter is defined as a representative value of the fiber diameter.

By the above action, the laminated fiber sheet 10 can develop/can have transferability of the liquid substance from the outer sheet 2 to the inner sheet 1 when the laminated fiber sheet 10 contains the liquid substance in the using method. Without limiting to a case where the liquid substance is applied from the face side of the outer sheet 2 (the other face side 10B) of the laminated fiber sheet 10, even in a state where the liquid substance is contained in the entire laminated fiber sheet 10, the liquid substance can be transferred to the inner sheet 1. In addition, the inner sheet 1 can act to push back or hold the liquid substance, which is about to return to the outer sheet 2, to the inner sheet 1.

The inner sheet 1 can become a liquid retaining layer by containing the liquid substance, and can form a uniform liquid film between the surface of the object and the inner sheet 1. And the inner sheet 1 can retain the liquid film for a relatively long time. This can be contributed by, in addition to the action described above, the inner sheet 1 being suitable (e.g., superior) in smoothness to the object surface by having a fiber structure made of ultra-fine fibers and suppressing the conventional unevenness of the fiber unit to be smaller.

Thus, the laminated fiber sheet 10 according to one or more embodiments of the present disclosure can form a uniform liquid film of the liquid substance (uniform wet state) in the inner sheet 1 by the cooperative action between the outer sheet 2 and the inner sheet 1. Then, the adhesion between the liquid substance and the object surface can be increased (e.g., dramatically) compared with the conventional one. And the state can be maintained for a relatively long time. This can allow the action of the liquid substance to exert on the object uniformly for a relatively long time.

This may cause the liquid substance to be unevenly distributed to the inner sheet 1 in a state where the laminated fiber sheet 10 contains the liquid substance and is moistened and maintains the state. As a result, in the laminated fiber sheet 10, the face side of the outer sheet 2 (the other face side 10B) can be put into a dry state, and the state can be sustained. That is, the laminated fiber sheet 10 can be put into a state where stickiness caused by the liquid substance is suppressed even if the face side of the outer sheet 2 (the other face side 10B) is touched.

The “uneven distribution” may be determined by comparing a ratio (mass ratio) obtained by dividing a content mass of the liquid substance in the inner sheet 1 by a sheet mass of the inner sheet 1 (not containing the liquid substance) with a ratio (mass ratio) obtained by dividing a content mass of the liquid substance in the outer sheet 2 by a sheet mass of the outer sheet 2 (not containing the liquid substance). For example, if the mass ratio of the inner sheet 1 is greater than the mass ratio of the outer sheet 2, it can be said that the liquid substance is unevenly distributed to the side of the inner sheet 1.

As this “uneven distribution”, it may be preferred that the inner sheet 1 is in a wet state and the outer sheet 2 is in a dry state. The “dry state” can mean a state where the mass ratio is equal to or less than 1.5. On the other hand, the “moistened state (wet state)” can mean a state where the mass ratio is more than 1.5.

### Method of Determining Uneven Distribution

(1) From the outer sheet 2 side, 0.15 g of liquid substance is applied over a 50 mm square laminated fiber sheet 10.
(2) The laminated fiber sheet 10 after the application is allowed to stand and recovered after 10 minutes.
(3) The inner sheet 1 and the outer sheet 2 are separated to measure each mass, and from a difference from the sheet mass before the application of the liquid substance, the content mass of the liquid substance is calculated.

Since the sheet mass cannot be measured by separating the inner sheet 1 and the outer sheet 2 before the application of the liquid substance, values converted from basis weights are employed.
(4) The ratio (mass ratio) obtained by dividing the content mass of the liquid substance in the inner sheet 1 by the sheet mass of the inner sheet 1 before the application of the liquid substance is calculated, and the mass ratio for the outer sheet 2 is calculated in the same manner. The mass ratio for the inner sheet 1 is compared with the mass ratio for the outer sheet 2, and the one having a larger value is determined to be in an uneven distribution state.

The laminated fiber sheet 10 of the present invention easily radiates heat because of ventilation spaces between the fibers of the dry outer sheet 2. In this respect, in a state where the liquid substance is applied, an endothermic reaction easily occurs when the liquid volatilizes. That is because the outer sheet 2 adjacent to the inner sheet 1 has a large fiber diameter while the inner sheet 1 maintains a wet state. And then the liquid substance is more likely to volatilize than the inner sheet 1 having a small fiber diameter. For example, immediately after the liquid substance is applied, the endothermic reaction may cause cool feeling. This phenomenon may become a factor that accelerates the transfer of heat under a condition of combining with the liquid substance. Further, the “uneven distribution” and “dry state” are even more likely to occur over time because a part of the liquid substance in the outer sheet 2 may volatilize along with the heat transfer.

As described above, the laminated fiber sheet 10 can create a persistent wet state of a liquid substance simultaneously while enabling stuffiness prevention of and stickiness suppression on the outer side by the action by the combination of the inner sheet 1 and the outer sheet 2.

From the perspective of enabling a clearer and faster development of the transferability of the liquid substance described above, for instance, in the laminated fiber sheet 10, a ratio (P2/P1) of a median fiber diameter (P2) of the outer sheet 2 to a median fiber diameter (P1) of the inner sheet 1 is preferably 1.5 or more, more preferably 10 or more, and further preferably 20 or more.

In addition, from the perspective of maintaining softness of the entire sheet, for instance, the ratio (P2/P1) is preferably 170 or less, more preferably 150 or less, and further preferably 100 or less.

From the perspective of developing the above-described transferability of the liquid substance and/or the uneven distribution of the liquid substance more clearly, and/or from the perspective of further increasing the adhesion with the object, as examples, the median fiber diameter (P1) of the inner sheet 1 is preferably 5 μm or less, more preferably 3 μm or less, further preferably 2 μm or less, and even further preferably 1 μm or less.

In addition, from the perspective of improving productivity, for instance, the median fiber diameter (P1) of the inner sheet 1 is preferably 0.3 μm or more, more preferably 0.4 μm or more, and further preferably 0.5 μm or more.

The outer sheet 2 preferably contains fibers having the median fiber diameter (P2) of 5 μm or more and 50 μm or less. By containing the fibers having the above median fiber diameter, the outer sheet 2 can further enhance the dry state and heat dissipation described above, provide sheet strength and/or rigidity of the entire laminated fiber sheet 10 without impairing the above-described adhesion property of the inner sheet 1, and increasing resistance against tearing and scraping. The outer sheet 2 can be obtained by setting the median fiber diameter as necessary.

From the perspective of maintaining the softness of the entire sheet, for instance, the median fiber diameter (P2) of the fibers contained in the outer sheet 2 is preferably 50 μm or less, further preferably 30 μm or less, and even further preferably 25 μm or less.

In addition, from the perspective of further increasing the resistance against tearing and scraping, for instance, the median fiber diameter (P2) of the fibers contained in the outer sheet 2 is preferably 5 μm or more, further preferably 10 μm or more, and even further preferably 15 μm or more.

In the laminated fiber sheet 10, a basis weight (M1) of the inner sheet 1 is preferably 1 g/m² or more and 8 g/m² or less. The basis weight (M1) of the inner sheet 1 within this range can allow maintaining the above-described softness, the adhesion to the object surface, and the uniformity of the liquid film of the liquid substance, which the inner sheet 1 has, and having strength without impairing the softness, the adhesion, and the uniformity. In addition, in the inner sheet 1 where the basis weight is limited to be in the above range, the number of ultra-fine fibers having the above-described median fiber diameter is reduced by that amount, and the number of fiber voids also decreases. This can suppress light scattering caused by the presence of the fiber voids and can make the inner sheet 1 transparent. In particular, since the fiber voids are filled with the liquid substance to form a uniform film when the liquid substance is applied, the light scattering may be more difficult to occur, and the transparency can become stronger.

From the perspective of further enhancing the above-described actions and/or from the perspective of further increasing the strength, for instance, the basis weight (M1) of the inner sheet 1 is preferably 1 g/m² or more, more preferably 1.5 g/m² or more, and further preferably 2 g/m² or more.

From the perspective of further increasing the softness and/or the adhesion to the object, for instance, the basis weight (M1) of the inner sheet 1 is preferably 8 g/m² or less, more preferably 6 g/m² or less, and further preferably 5 g/m² or less.

In the laminated fiber sheet 10, a basis weight (M2) of the outer sheet 2 is preferably 8 g/m² or more and 50 g/m² or less. The basis weight (M2) of the outer sheet 2 within this range further enhances the dry state and the heat dissipation of the outer sheet 2 and can further increase the strength of the entire laminated fiber sheet 10, which can allow further increasing the resistance against tearing and scraping. In addition, in the outer sheet 2 where the basis weight is set to the above range, the number of fibers having the above-described median fiber diameter is reduced. This can make the outer sheet 2 transparent, and in particular, the transparency can be regarded as high, for instance, when a preparation is applied.

From the perspective of further increasing the strength of the entire laminated fiber sheet 10, for instance, the basis weight (M2) of the outer sheet 2 is preferably 8 g/m² or more, more preferably 15 g/m² or more, and further preferably 17 g/m² or more.

From the perspective of further enhancing the dry state and the heat dissipation of the outer sheet 2, and/or from the perspective of not impairing the softness and/or the adhesion to a skin surface of the inner sheet 1, for instance, the basis weight (M2) of the outer sheet 2 is preferably 50 g/m² or less, more preferably 40 g/m² or less, and further preferably 30 g/m² or less.

From the perspective of further improving the above-described transferability of the liquid substance, for instance, the uneven distribution of the liquid substance to the inner sheet 1, and the dry state and the heat dissipation of the outer sheet 2, a ratio (M2/M1) of the basis weight (M2) of the outer sheet 2 to the basis weight (M1) of the inner sheet 1 is preferably 1 or more, more preferably 1.6 or more, and further preferably 2 or more.

From the perspective of maintaining the above-described softness and/or the adhesion to the object surface, which the inner sheet 1 has, for instance, the ratio (M2/M1) is preferably 50 or less, more preferably 34 or less, and further preferably 25 or less.

From the perspective of properly forming the liquid film of the liquid substance, for instance, in the laminated fiber sheet 10, a thickness of the inner sheet 1 is preferably 3 μm or more, more preferably 4 μm or more, and further preferably 5 μm or more.

From the perspective of causing the above-described softness and/or the adhesion to the object surface, which the inner sheet 1 has, for instance, the thickness of the inner sheet 1 is preferably 50 μm or less, more preferably 40 μm or less, and further preferably 30 μm or less.

From the perspective of further increasing the strength of the entire laminated fiber sheet 10, for instance, a thickness of the outer sheet 2 is preferably 10 μm or more, more preferably 30 μm or more, and further preferably 50 μm or more.

From the perspective of further enhancing the dry state and/or the heat dissipation of the outer sheet 2, and/or from the perspective of not impairing the softness and/or the adhesion to a skin surface and the like of the inner sheet 1, for instance, the thickness of the outer sheet 2 is preferably 500 μm or less, more preferably 300 um or less, and further preferably 200 μm or less.

From the perspective of enhancing integrity (e.g., further enhancing) of the inner sheet 1 and the outer sheet 2, for instance, in the laminated fiber sheet 10, the inner sheet 1 and the outer sheet 2 preferably contain materials of the same family. The material of the same family can be a material having a polymer structure in which repeating units (monomer structures) are identical. Specifically, the material of the same family may be one selected from an olefin-based resin, a diene-based resin, a urethane-based resin, and a copolymer thereof.

By containing the materials of the same family by the inner sheet 1 and the outer sheet 2, the molecular structures of resin components can be similar to one another, and compatibility of both fibers can be high. Therefore, the inner sheet 1 and the outer sheet 2 can be fused strongly, which can enhance the integrity of the laminated fiber sheet 10.

The laminated fiber sheet 10 preferably has stretchability. This can allow the laminated fiber sheet 10 to enhance fitting performance and/or followability relative to the object and/or to further increase the adhesion of the above-described liquid film of the liquid substance formed in the inner sheet 1 relative to the object.

For the stretchability, the inner sheet 1 and the outer sheet 2 preferably has stretchability by containing elastomers of the materials of the same family selected from the above-described olefin-based resin, diene-based resin, urethane-based resin, and copolymer thereof.

### Method of Measuring Inner Sheet 1 and Outer Sheet 2 Containing Materials of the Same Family

For the method of measuring the inner sheet 1 and the outer sheet 2 containing the materials of the same family, delamination may be performed between respective sheets to measure resin compositions contained in the respective sheets. The resin compositions are provided for various analyses, such as nuclear magnetic resonance (NMR) analysis and infrared spectroscopy (IR) analysis, and structures of molecular skeletons and functional group structures at the end of the molecular structures are identified based on the position of each signal and spectrum obtained by these analyses. Accordingly, the type of contained resin is identified and specified for each sheet. The resin composition specified for each sheet is compared to determine whether the materials of the same family are contained.

In the laminated fiber sheet 10 according to one or more embodiments according to the present disclosure, both the inner sheet 1 and the outer sheet 2 adjacent to one another can have relatively high integrity while having stretchability, which can make it difficult for delamination to occur, for instance, at an interface between the inner sheet 1 and the outer sheet 2 during extension and contraction. Suppression of the delamination can generate or result in the sheet strength and/or rigidity in the entire laminated fiber sheet 10, and firmness appears in whole. Then, the inner sheet 1 can extend and contract together with the outer sheet 2. This can allow the laminated fiber sheet 10 as a whole to have durability to withstand repeated extending/contracting motions. That is, the laminated fiber sheet 10 according to one or more comments of the present disclosure can extend and contract in a state where the inner sheet 1 and the outer sheet 2 have a high integrity and the overall sheet strength and/or rigidity can be increased. Therefore, in an extending/contracting behavior of the laminated fiber sheet 10, twist and scraping of the inner sheet 1 can be difficult to occur. In addition, fiber loss, for instance, caused by scraping in the inner sheet 1, can be suppressed.

Thus, in the laminated fiber sheet 10, the stretchability as well as the sheet strength can be suitable (e.g., improve as a whole), and even when the stretchability develops, the resistance against tearing and scraping can be increased. Then, the extension and contraction of the entire laminated fiber sheet 10 can allow the inner sheet 1 to follow shapes of the object surface and various changes (movements) and/or to suitably (e.g., partially or fully) demonstrate at least the above-described adhesion property even under the changes.

The laminated fiber sheet 10 can have rigidity by the integrity of the inner sheet 1 and the outer sheet 2, which can enhance handling performance. That is, the firmness can be obtained, for instance, more than a case of a fiber sheet having only the inner sheet 1 made of ultra-fine fibers (e.g., FIGS. 2(A) and 2(B)), and an effect of making handling suitable (e.g., easier) can also provided. For example, when one laminated fiber sheet 10 is about to be taken out and used from a state where many laminated fiber sheets 10 are stacked or a state where many laminated fiber sheets 10 are stored in a packaging bag, a container, or the like (for example, affixed to the object surface), the firmness may affect (e.g., significantly) the ease of taking out. In addition, when the laminated fiber sheet 10 according to one or more embodiments of the present disclosure is taken out, it may be more difficult to curl up compared to the fiber sheet having only the inner sheet 1 (for example, FIG. 2(A)), which can enhance handling performance. This can allow affixing to an object, such as a skin surface, (e.g., neatly, without wrinkles, etc.).

From the perspective of enhancing the integrity of the inner sheet 1 and the outer sheet 2 and/or enhancing the stretchability of the laminated fiber sheet 10 including the inner sheet 1 and the outer sheet 2, for instance, the above materials of the same family are preferably an olefin-based resin. Using the olefin-based resin can make it difficult for the inner sheet 1 and the outer sheet 2 to delaminate when the laminated fiber sheet 10 is extended and contracted.

From a similar perspective, for instance, the inner sheet 1 preferably contains an olefin-based resin with low crystallinity. The “low crystallinity” can mean that crystallinity is 10.5% or less and can be measured by the following method. By containing an olefin-based resin with low crystallinity by the inner sheet 1, extension and contraction of the inner sheet 1 itself can be performed (e.g., easily).

When the inner sheet 1 contains an olefin-based resin with low crystallinity, a mass proportion of the olefin-based resin with low crystallinity occupied in the inner sheet 1 is preferably 70 mass % or more, more preferably 80 mass % or more, further preferably 90 mass % or more, further preferably 92 mass % or more, and even more preferably 94 mass % or more.

### Method of Measuring Inner Sheet 1 Containing Olefin-Based Resin with Low Crystallinity

The resin composition constituting the inner sheet 1 is provided for various analyses, such as NMR analysis and IR analysis, and the structure of the molecular skeleton and the functional group structure at the end of the molecular structure are identified based on the position of each signal and spectrum obtained by these analyses. Accordingly, the type of resin contained is identified and specified.

Next, for the crystallinity, differential scanning calorimetry is used to be able to confirm being low crystalline. The total heat of fusion obtained when the temperature of a specimen is raised is divided by the heat of fusion of a perfect crystal. When this value is less than 10.5%, it is determined that an olefin-based resin with low crystallinity is contained.

From the perspective of enhancing the integrity of the inner sheet 1 and the outer sheet 2 and enhancing the stretchability of the laminated fiber sheet 10 including the inner sheet 1 and the outer sheet 2, for instance, the above materials of the same family include, for example, the following specific examples.

That is, examples of the olefin-based resin include polypropylene (hereinafter referred to as PP), polyethylene, and the like.

Examples of the olefin-based resin with low crystallinity can include a-olefin and the like including polypropylene with stereoregularity controlled (polypropylene polymerized with stereoregularity controlled by a metallocene catalyst).

Examples of the diene-based resin can include polybutadiene, polyisoprene, and the like.

Examples of the urethane-based resin can include polyurethane and the like.

Examples of the copolymer of the olefin-based resin, diene-based resin, and urethane-based resin can include an ethylene-propylene copolymer, an ethylene-1-butene copolymer, an ethylene-1-octen copolymer, a propylene-1-butene copolymer, a propylene-1-octen copolymer, an ethylene-isoprene copolymer, an ethylene-butadiene copolymer, a propylene-isoprene copolymer, a propylene-butadiene copolymer, an ethylene-propylene-isoprene copolymer, an ethylene-propylene-butadiene copolymer, and the like.

In the laminated fiber sheet 10 according to one or more embodiments of the present disclosure, both the inner sheet 1 and the outer sheet 2 preferably have stretchability and are integrated. Here, “have stretchability” can be a property that allows extending and returns to a length of 125% or less of an original length when force is released from a state of extending 50% of the original length (having a length of 150% of the original length).

In the laminated fiber sheet 10 according to one or more embodiments of the present disclosure, it is preferred that an extension percentage in one direction Y (in other words, Y-direction) in a plane direction is 20% or more and 100% or less, and an extension percentage in a direction X (in other words, X-direction) perpendicular to the one direction Y is 0% or more and 30% or less. By causing a difference in extension percentage in these two directions perpendicular to one another, the laminated fiber sheet 10 can have a directionality of stretchability (e.g., one or more directions of stretchability, only one direction of stretchability, etc.). The one direction Y and the direction X perpendicular to the one direction Y can be determined as necessary according to the purpose of use of the laminated fiber sheet 10. For example, a longitudinal direction of the laminated fiber sheet 10 may be the one direction Y, and a width direction may be the direction X perpendicular to the one direction Y. In this case, the longitudinal direction of the laminated fiber sheet 10 is preferably aligned along an extending/contracting direction required by an article to be applied.

### Method of Measuring Extension Percentage

The above extension percentage in the laminated fiber sheet 10 is extension of a test specimen at a tensile load of 1.2 N indicated by a proportion (%) relative to a grip distance of 50 mm. The test specimen has a width of 20 mm and a length of 70 mm and is fixed at the grip distance of 50 mm. More specifically, the length of a grip interval of the test specimen before extension testing is set as LO, and the length of the grip interval of the test specimen at the time of extension at break is set as LB, and the extension percentage can be obtained as the extension percentage (%)=100×(LB−L0)/L0. The measurement is performed along any two directions perpendicular to one another within the sheet plane of the laminated fiber sheet 10, for example, the two directions that are the longitudinal direction and the width direction perpendicular to the longitudinal direction.

By having the directionality of stretchability by the above difference in extension percentage, the laminated fiber sheet 10 can be optimally controlled to achieve stretchability according to the purpose of use and reduce excessive extension in a direction that does not require stretchability or excessive load in association with the extension and contraction. This can allow suppressing twist, scraping, and/or tearing of the inner sheet 1 (e.g., more effectively).

For example, when the laminated fiber sheet 10 is made into gloves, it may be preferred that the stretchability in a length direction of fingers is large and the stretchability in a width direction perpendicular to the length direction is small. This can allow providing a glove having high adhesion to user's fingertips and interdigital webs.

When the laminated fiber sheet 10 is made into gloves, for instance, it may also be preferred that the stretchability in the length direction of fingers is small and the stretchability in the width direction perpendicular to the length direction is large. This can allow providing gloves having high adhesion according to a thickness of user's fingers when the gloves in which the laminated fiber sheet 10 is used are fitted.

From the perspective of further enhancing the above-described actions, for instance, an extension percentage (T1) in the one direction Y of the laminated fiber sheet 10 is preferably 20% or more, more preferably 25% or more, and further preferably 30% or more.

From the perspective of shaping the laminated fiber sheet 10 into any shape to be fitted, for instance, the extension percentage (T1) in the one direction Y in the laminated fiber sheet 10 is preferably 100% or less, more preferably 80% or less, and further preferably 60% or less.

From the perspective of continuously conveying the laminated fiber sheet 10, for instance, an extension percentage (T2) in the direction X perpendicular to the one direction Y of the laminated fiber sheet 10 is preferably 0% or more.

From the perspective of continuously conveying the laminated fiber sheet 10, for instance, the extension percentage (T2) in the direction X perpendicular to the one direction Y in the laminated fiber sheet 10 is preferably 30% or less, more preferably 25% or less, and further preferably 20% or less.

From the perspective of further clarifying the directionality of stretchability of the laminated fiber sheet 10 and/or suppressing twist, scraping, and/or tearing of the inner sheet 1 (e.g., more effectively), for instance, an absolute value |T1−T2| of a difference between the extension percentage (T1) in the one direction Y of the laminated fiber sheet 10 and the extension percentage (T2) in the direction X perpendicular to the one direction Y is preferably 0 or more, more preferably 10 or more, and further preferably 20 or more.

From the perspective of enhancing a fitting property of the laminated fiber sheet 10 to an object (such as skin), for instance, the absolute value |T1−T2| of the difference is preferably 70 or less, more preferably 60 or less, and further preferably 50 or less. This can make the gloves, for example, when the laminated fiber sheet 10 is applied to gloves, easy to fit by balancing the extension percentages in two directions perpendicular to one another in the gloves.

The above directionality of stretchability in the laminated fiber sheet 10, according to one or more embodiments of the present disclosure, can be specified (e.g., mainly, only) by the outer sheet 2 rather than the inner sheet 1. Since the inner sheet 1 can contain the above-described ultra-fine fibers in which, for example, a resin solution or resin molten liquid is directly spun and deposited using the electrospinning method, the directionality of stretchability is difficult to form. Therefore, the directionality of stretchability of the laminated fiber sheet 10 may be provided (e.g., mainly, etc.) by the outer sheet 2. In addition, by specifying the above directionality of stretchability in the laminated fiber sheet 10 by (e.g., mainly, etc.) the outer sheet 2, the outer sheet 2 can be optimally controlled so as to stop extending before the inner sheet 1 tears when the laminated fiber sheet 10 extends. The directionality of stretchability by the outer sheet 2, for example, can allow forming the outer sheet 2 that may not have stretchability in a conveying direction but can have stretchability in a perpendicular direction by extending in the conveying direction and cooling to solidify in the course of conveyance.

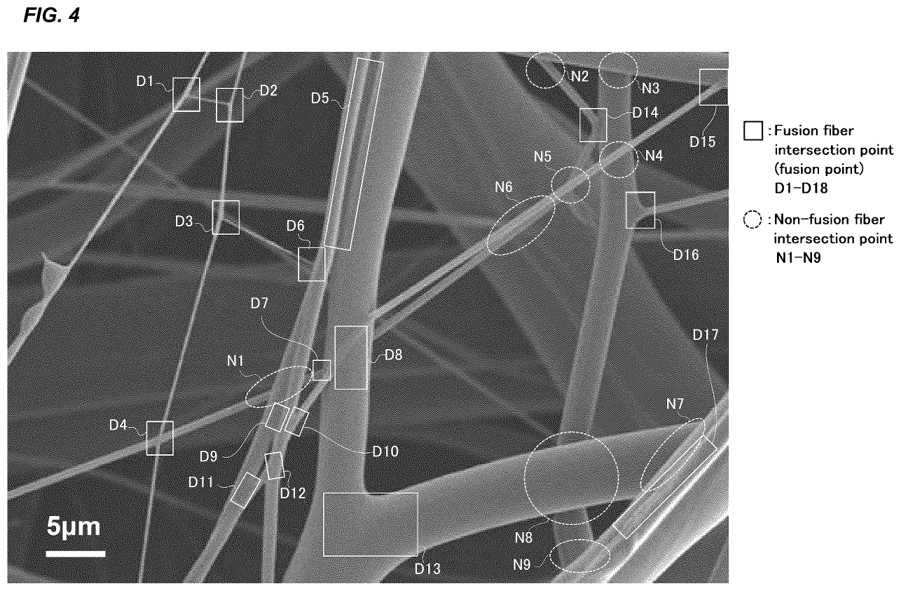

From the perspective of further increasing the strength of the inner sheet 1 itself in the laminated fiber sheet 10, for instance, a proportion of the number of fusion bonding points (for example, the fusion bonding point 31 at an intersection point of ultra-fine fibers 11, 11 in FIG. 3, and enclosed sections of reference numerals D4, D14, and D1 in FIG. 4) of the inner sheet 1 occupied in the number of fiber intersection points in a visual field of 0.128 mm×0.096 mm of an SEM image is preferably 50% or more, and more preferably 70% or more. This can ensure that one by one of the ultra-fine fibers of the inner sheet 1 is fixed at the intersection points, which can increase the strength. That is, the integrity of the inner sheet 1 itself can be strengthened. For example, when the surface of the inner sheet 1 at the one face side 10T of the laminated fiber sheet 10 is traced with a finger, fluff of the fibers may be unsuitable (e.g., difficult) to stand up, and dropping-off of the fibers may be suppressed. In addition, since a bonded area can be suppressed at the fusion bonding points at the fiber intersection points, an excessive increase in rigidity of the inner sheet 1 itself can be suppressed and softness can be retained, for instance, compared with planar fusion bonding, and the above-described adhesion property can be developed (e.g., easily).

### Proportion of the Number of Fusion Bonding Points Occupied in the Number of Fiber Intersection Points

Operations similar to (1), (2), and (3) in the above-described (Method of Measuring Median Fiber Diameter) chapter are performed to obtain SEM observation images with an observation magnification of 1 000 times for a fiber layer to be measured. The same sample is measured at a total of five locations by changing the observation location. A total of five observation images per location are obtained with a focus on the sample surface side. An image quality is a width of 0.128 mm and a height of 0.096 mm. From the obtained observation images, the fiber intersection points are marked using the image analysis software (WinROOF2015, manufactured by MITANI CORPORATION), and the number of fusion bonding points at the fiber intersection points is recorded. The proportion of the number of fusion bonding points occupied in the total number of fiber intersection points can be calculated by the following formula (I). This evaluation is performed by two people, an observer and a recorder. The number of fiber intersection points is aggregated by the marking process by the hands of the observer on the above image analysis software. For the fusion bonding points, the recorder aggregates the fiber intersection points can be defined as the fusion bonding points by the observer according to the definition of the fusion bonding point below.

$$Proportion of fusion bonding points [ \% ] = ( number of fusion bonding points ) / ( total number of fiber intersection points ) \times 100 \qquad ( formula ( I ) )$$

The definitions of the fiber intersection point and the fusion bonding point of the fiber intersection point can be defined as follows.

### Fiber Intersection Point

In the SEM observation image of a fiber layer to be measured, the image itself is enlarged by a factor of three (300%) on a monitor of 410 mm×260 mm. In this case, fibers being in focus in a range where a width between boundary lines of a fiber shape (fiber width observed in the observation image) does not exceed 1 mm are selected. A location at which the fibers intersect, a location at which they do not intersect but are touched (in contact), and a location at which a single fiber branches in the middle, each are defined as a fiber intersection point (for example, the enclosed sections of the reference numerals D4, D14, and D1 in FIG. 4). When those in which two or more fibers appearing in the image come in contact in the longitudinal direction in whole or part of the imaged area to form a fiber bundle are seen in the observation image, they are also defined as fiber intersection points (for example, an enclosed section of a reference numeral D5 in FIG. 4). For example, it is set that, when there are locations where four fibers are aligned without gaps in the observation image, the number of fiber intersection points is defined as three. Due to the characteristics of the fiber layer, in the SEM observation, there is a depth caused by the thickness of the fiber layer, which includes a side close to a detector (a surface side in a specimen thickness direction) and a side far from the detector (a side of a sample stage for specimen in the specimen thickness direction). Therefore, even though fibers appear to intersect apparently in the observation image, the fibers are not in contact with one another in some cases. As described above, when the image itself is enlarged by a factor of three (300%) on a monitor of 410 mm×260 mm, a plurality of fibers that are in focus in a range where the width of the boundary lines of the fiber shape does not exceed 1 mm are assumed to have an equal positional relationship in the thickness direction (depth) of the specimen. The intersection point of the fibers thus selected can be defined as a fiber intersection point in accordance with the above-descried definition.

### Fusion Bonding Point of Fiber Intersection Point

Among the intersection points such as defined above, those defined as the fusion bonding points of the fiber intersection points can be (i) a location where a boundary line of two or more related fibers at the intersection point is not clearly recognized (for example, an enclosed section of a reference numeral D4 in FIG. 4) and/or (ii) a location where, of one or more related fibers at the intersection point, a width between boundary lines of the fiber at the intersection point demonstrates expansion compared with a width between the boundary lines at a point other than the intersection point (for example, an enclosed section of a reference numeral D16 in FIG. 4). When the sheet is in a laminated state, delamination can be performed between the fiber layers, and the fiber layer to be measured can be taken out and provided for the above work.

The laminated fiber sheet 10 can be regarded as having the integrity of the inner sheet 1 and the outer sheet 2 and suitable strength (e.g., high). The strength of the integrity preferably can withstand the stretchability such as described above. This strength can be indicated as breaking strength. The breaking strength according to one or more embodiments of the present disclosure can be in the following range in the two directions X and Y that indicate the above-described extension percentages from a relationship with the above-described stretchability and a relationship with the resistance against scraping and tearing of the inner sheet 1 in the extending/contracting behavior. From the perspective of obtaining the resistance against extension, for instance, the breaking strength in the Y-direction is preferably 1 N/20 mm or more, more preferably 2 N/20 mm or more, and further preferably 2.5 N/20 mm or more.

From the perspective of facilitating the conveyance of the laminated fiber sheet 10, for instance, the breaking strength in the X-direction is preferably 1

N/20 mm or more, more preferably 3 N/20 mm or more, and further preferably 5 N/20 mm or more.

### Method of Measuring Breaking Strength of Laminated Fiber Sheet 10

For the breaking strength in the laminated fiber sheet 10, a test specimen of a width of 20 mm and a length of 70 mm is fixed at a grip distance of 50 mm and extended at a tensile speed of 300 mm/min, and the maximum load until the test specimen breaks is defined as the breaking strength. The measurement temperature is 23° C.

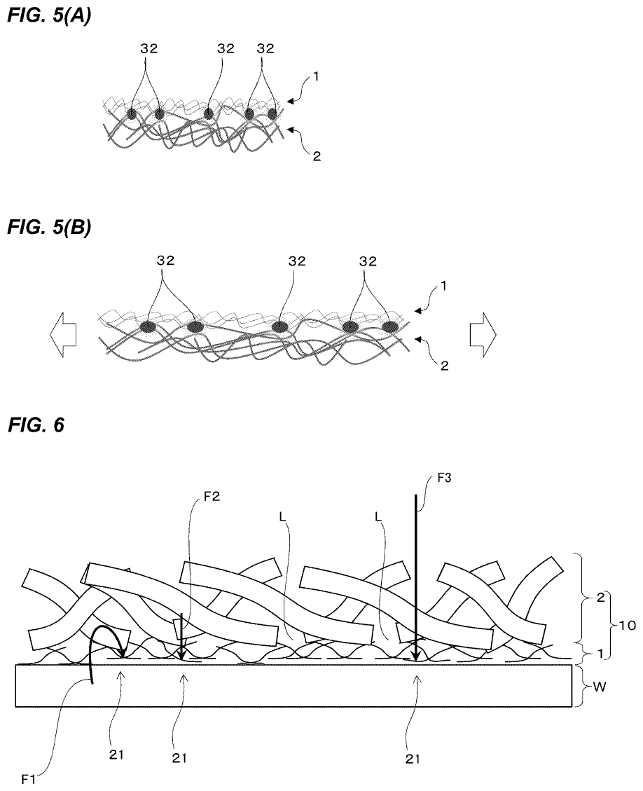

In the laminated fiber sheet 10, at the interface of the inner sheet 1 and the outer sheet 2, both fibers can be fused, for instance, from the perspective of improving the integrity (for example, fusion bonding points 32 of FIG. 5(A)). In addition, both fibers at the interface can be joined by the fusion bonding point at the intersection point of both fibers with fiber shapes retained, for instance, in terms of suppressing an excessive increase in rigidity of the laminated fiber sheet 10.

The term fusion bonding can mean that the resin components contained in the inner sheet 1 and the outer sheet 2 are melted by heat and bonded (for example, heat bonding). Since the inner sheet 1 and the outer sheet 2 contain the materials of the same family with high compatibility as described above, the fusion bonding at the interface can be suitable (e.g., stronger). This can allow the integrity of the inner sheet 1 and the outer sheet 2 to further increase while maintaining the softness on the one face side 10T of the inner sheet 1 itself and the adhesion to a skin surface and the like. In particular, the delamination at the interface between the inner sheet 1 and the outer sheet 2 can be more effectively suppressed during the extending/contracting behavior of the laminated fiber sheet 10, and the followability of both layers can become more satisfactory (for example, from the state of FIG. 5(A) to the state of FIG. 5(B)). This can further ensure the development of the above-described transferability of the liquid substance from the outer sheet 2 to the inner sheet 1. In addition, when the inner sheet 1 and the outer sheet 2 are integrated at the fusion bonding point at the intersection point of both fibers, voids between the fibers at the interface between both sheets may be retained (e.g., easily), and the development of the above-described liquid transferability can be further reliably smooth. Moreover, further improvement of the integrity can make the above-described handling performance related to the laminated fiber sheet 10 more satisfactory.

From the perspective of further improvement of the above actions, for instance, the strength of fusion bonding at the interface is preferably 0.01 N/20 mm or more, more preferably 0.1 N/20 mm or more, and further preferably 0.2 N/20 mm or more.

In addition, the strength of fusion bonding at the interface can be 10 N/20 mm or less, according to one or more embodiments of the present disclosure.

In the above range, the laminated fiber sheet 10 can be integrated.

### Method of Measuring Strength of Fusion Bonding at Interface between Inner Sheet 1 and Outer Sheet 2

The measurement is performed by the following means.

Adhesive tape is affixed to the surface of the inner sheet 1 of the laminated fiber sheet 10, and the tape is peeled off in a T-shape with a tensile tester. This allows measuring the strength of fusion bonding of both fibers at the interface between the inner sheet 1 and the outer sheet 2. The more the number of fusion bonding points at the interface between the inner sheet 1 and the outer sheet 2, the more resistive power increases at the time of peeling off in a T-shape.

After adhesive tape (adhesive capacity: 4.4 N/10 mm) is affixed to the surface side of the inner sheet 1, a test specimen with a width of 20 mm and a length of 100 mm is prepared. A tape end portion on the longitudinal direction side is peeled off, and the tape end portion and the laminated fiber sheet 10 are pulled at a grip distance of 50 mm and a tensile speed of 100 mm/min to measure a test force that causes the tape to peel off from the laminated fiber sheet 10.

Out of the obtained test forces, the average value of the test forces within a travel distance of a grip tool of 30 mm or more and 80 mm or less from the initial stage is determined as the strength of fusion bonding, that is, a fusion bonding force.

The fusion bonding point at the fiber intersection point in the inner sheet 1 and the fusion bonding point of both fibers at the interface between the inner sheet 1 and the outer sheet 2 described above can be suitably formed by containing the materials of the same family by the inner sheet 1 and the outer sheet 2. Then, the fusion bonding points can be formed as follows.

On the surface of the outer sheet 2, a resin solution or resin molten liquid using the above-described material of the same family is discharged from a nozzle, for example, by the electrospinning method. Spun fibers formed by discharging (constituent fibers of the inner sheet 1) are collected on the outer sheet 2 in a state of having high fluidity at a high temperature. This can allow collecting the spun fibers in a state where they are fused together at the intersection points, and the spun fibers and the fibers of the outer sheet 2 are integrated in a state of being fused. Collecting such spun fibers can form the inner sheet 1 in which a network having the above-described fusion bonding points at the fiber intersection points is formed. Simultaneously, the fusion bonding points of both fibers at the interface between the inner sheet 1 and the outer sheet 2 can be formed. The temperature at which a state in which the above spun fibers have fluidity is brought is appropriately set according to the resin raw materials used. For example, when a low-crystalline PP resin is used as the resin raw material of the inner sheet 1, the spun fibers are preferably collected in a range of 40° C. or more and 70° C. or less, at which it changes to a rubber state. Additionally, such a temperature is preferably maintained by heating the outer sheet 2 that is a receiving pan for the collection.

In addition to the above method, the above-described fusion bonding points can be formed by heat sealing. From the perspective of avoiding heat shrinkage of fiber elastomers and film formation caused by melting of resin, for instance, as described above, fusion bonding during spinning with less time and frequency of exposure to heat may be more preferable.

Next, the following describes a method of using the laminated fiber sheet 10 of one or more embodiments of the present disclosure.

In the method of using the laminated fiber sheet 10, the above-described laminated fiber sheet 10 is moistened with the liquid substance.

Specifically, a step of applying the laminated fiber sheet 10 to an object with the inner sheet 1 facing toward the object and a step of applying the liquid substance are included.

The laminated fiber sheet 10 can develop the transferability of the liquid substance from the outer sheet 2 to the inner sheet 1 by steps such as the two steps.

Specifically, the above-described two steps can include cases in which they are performed in any of procedures (I) to (III) below. In any procedure, the laminated fiber sheet 10 can develop the transferability of the liquid substance from the outer sheet 2 to the inner sheet 1 and can create a wet state of the liquid substance in the inner sheet 1 (in particular, the surface of the inner sheet 1 opposed to the object). Then, the outer sheet 2 can be put into a dry state. The amount of application of the liquid substance in the step of applying the liquid substance can be within the range indicated as the “amount of application of the liquid substance to enter a wet state” described above.
(I) After the liquid substance is applied to the object, the laminated fiber sheet 10 is applied with the inner sheet 1 facing toward the object.
(II) After the liquid substance is applied to the laminated fiber sheet 10, the laminated fiber sheet 10 is applied with the inner sheet 1 facing toward the object.
(III) After the laminated fiber sheet 10 is applied with the inner sheet 1 facing toward the object, the liquid substance is applied to the laminated fiber sheet 10.

The development of the transferability of the liquid substance from the outer sheet 2 to the inner sheet 1 described above may include not only transferring the liquid substance present in the outer sheet 2 to the inner sheet 1, but also pushing the liquid substance that attempts to flow backward from the inner sheet 1 to the outer sheet 2 or the liquid substance that flows backward back to the inner sheet 1. FIG. 6 schematically illustrates an exemplary transfer of a liquid substance in a state where the laminated fiber sheet 10 is applied to an object W. Arrows F1, F2, and F3 indicate only rough directions of the transfer of the liquid substance, and the actual transfer form of the liquid substance may be more complex than them. The respective transfer directions of the liquid substance indicated by the arrows F1, F2, and F3 are illustrated as those at different locations and in separate procedures for convenience of explanation. However, in practice, the transfer directions can happen at the same location and in the same procedure, and in terms of time, they can happen at different times, not necessarily at the same time.

In the procedure (I), before the laminated fiber sheet 10 is applied with the inner sheet 1 facing toward the object, the liquid substance is applied to the object beforehand. A method of applying the liquid substance to the object can be performed by various methods normally used, such as spraying. In this case, the liquid substance initially exists at the interface between the inner sheet 1 and the object. At this time, the inner sheet 1 absorbs the liquid substance by its own extremely strong capillary force. Since the inner sheet 1 and the outer sheet 2 can be integrated and the inner sheet 1 is an ultra-thin layer of the ultra-fine fibers described above, the liquid substance may transfer (e.g., gradually) to the outer sheet 2. However, in such a wet state, the laminated fiber sheet 10 can act to push the liquid substance that transfers from the inner sheet 1 to the outer sheet 2 back to the inner sheet 1 by the above-described high liquid retention capacity of the ultra-fine fibers of the inner sheet 1 in addition to the above-described capillary force (for example, the arrow F1 in FIG. 6). This can cause the liquid substance to remain in the inner sheet 1.

In the procedure (II), the laminated fiber sheet 10 is in a wet state where the liquid substance is applied in advance. Examples of the application of the liquid substance can include a method of dipping the laminated fiber sheet 10 in a solution of the liquid substance or a method of using coating methods, such as spraying. In this case, a subject to which the liquid substance is applied may be both the inner sheet 1 and the outer sheet 2 of the laminated fiber sheet 10, or may be any one of the inner sheet 1 and the outer sheet 2. In any case, the laminated fiber sheet 10 in a wet state transfers the liquid substance from the outer sheet 2 to the ultra-thin inner sheet 1 (for example, the arrow F2 in FIG. 6) by the above-described high liquid retention capacity of the ultra-fine fibers of the inner sheet 1 in addition to the above-described capillary force so as to cause the liquid substance to remain in the inner sheet 1.

In the procedure (III), the liquid substance is applied to the laminated fiber sheet 10 applied to the object.

More specifically, in a state where the face side of the inner sheet 1 (one face side 10T) is brought into contact with the object, the liquid substance is applied to the laminated fiber sheet 10 from the face side of the outer sheet 2 (the other face side 10B). In this case, the laminated fiber sheet 10 can also transfer the liquid substance soaked from the outer side to the inner sheet 1 (for example, the arrow F3 in FIG. 6) by the above-described high liquid retention capacity of the ultra-fine fibers of the inner sheet 1 in addition to the above-described capillary force so as to cause the liquid substance to remain in the inner sheet 1.

As described above, even when the above-described two steps are performed using any of the procedures (I) to (III), the laminated fiber sheet 10 can form a uniform liquid film (uniform wet state) of the liquid substance in the inner sheet 1 by the cooperative action between the outer sheet 2 and the inner sheet 1, can increase (e.g., dramatically) the adhesion between the liquid substance and the object surface compared with the conventional one, and can maintain the state for a suitable amount of time (e.g., long time). This can allow the action of the liquid substance to exert on the object uniformly for a long time. As a result, the liquid substance can be allowed to be unevenly distributed to the inner sheet 1, and the state can be easily maintained. In addition, in the laminated fiber sheet 10, the face side of the outer sheet 2 (the other face side 10B) can be put (e.g., easily) into a dry state, and the state can be sustained (e.g., easily). That is, stickiness caused by the liquid substance can be suppressed even if the face side of the outer sheet 2 (the other face side 10B) of the laminated fiber sheet 10 is touched.

In the method of using the laminated fiber sheet 10, the inner sheet 1 may be regarded as tearable because it contains ultra-fine fibers and is ultra-thin, and friction may be as little as possible when applied to an object. From this perspective, for instance, it is preferred that the liquid substance is applied after the laminated fiber sheet 10 is applied to an object as described in the above (III), rather than that the liquid substance is first applied to an object or the laminated fiber sheet 10 and then the laminated fiber sheet 10 is applied to the object as described in the above procedures (I) and (II). In particular, when the laminated fiber sheet 10 is glove-shaped and is fitted to human fingers, the resistance caused by the liquid substance can be avoided to smoothly perform the fitting, and the liquid substance can be delivered (e.g., quickly) to the inner sheet 1 in the fitted state. In addition, since a uniform liquid film can be formed in the inner sheet 1 and the face side of the outer sheet 2 (the other face side 10B) can enter a dry state as described above, when the laminated fiber sheet 10 is fitted to human fingers, it can be possible to continue various daily activities, including the operation of electronic devices, such as a smartphone, with it fitted.

In the method of using the laminated fiber sheet 10, as illustrated in FIG. 6, contact regions 21 and void regions (non-contact regions) L preferably exist in a mixed manner at the interface between the outer sheet 2 and the inner sheet 1 that have different median fiber diameters. In the contact region 21, the constituent fibers of the outer sheet 2 come into contact with the fiber layer of the inner sheet 1. In the void region (non-contact region) L, the constituent fibers of the outer sheet 2 are not in contact with the fiber layer of the inner sheet 1. This can cause the development of the transferability of the liquid substance from the outer sheet 2 to the inner sheet 1, the formation of a uniform liquid film of the liquid substance in the inner sheet 1, and the achievement of a dry state in the outer sheet 2, which are described above, to be more satisfactory. In addition, when the inner sheet 1 and the outer sheet 2 are joined at the fusion bonding point of the fiber intersection point as described above, a certain strength is obtained, there may be no lifting at the interface, and the capillary force can be suitably (e.g., more easily) developed. Therefore, a uniform wet state in the inner sheet 1 can suitably occur (e.g., more easily), which can improve the retention of a liquid.

The method of using the laminated fiber sheet 10 can have a step of further applying the liquid substance which has been applied once from the face side of the outer sheet 2 of the laminated fiber sheet 10. That is, while the laminated fiber sheet 10 is applied to the object, the liquid substance can be reapplied. In this case, the liquid substance can be transferred and may be unevenly distributed to the inner sheet 1 by the above-described high liquid retention capacity of the ultra-fine fibers of the inner sheet 1 in addition to the above-described capillary force, and the outer sheet 2 can be put into a dry state. Therefore, the step can be performed a suitable number of times.

In the method of using the laminated fiber sheet 10, even when the liquid substance is contained in the laminated fiber sheet 10, the fibers constituting the laminated fiber sheet 10 can keep the fiber shape. This can become possible by the inner sheet 1 and the outer sheet 2 being insoluble to the liquid substance as described above. This can cause the above-described action on the liquid substance in the fiber structure of the laminated fiber sheet 10 and the uneven distribution of the liquid substance to the inner sheet 1 to be more pronounced. In addition, the further application of the liquid substance described above can be performed more properly. Moreover, handleability of the laminated fiber sheet 10 can also improve.

In the method of using the laminated fiber sheet 10, a proportion of a content basis weight of the liquid substance occupied in a basis weight of the laminated fiber sheet 10 can be preferably 50% or more. This can make it suitable (e.g., easier) to form a uniform film of the liquid substance and can cause the development of the transferability of the liquid substance from the outer sheet 2 to the inner sheet 1, the formation of a uniform liquid film of the liquid substance in the inner sheet 1, and the achievement of a dry state in the outer sheet 2, which are described above, to be suitable (e.g., more satisfactory).

### Method of Measuring Ratio of Content Basis Weight of the Liquid Substance to Basis Weight of the Laminated Fiber Sheet

(1) A mass of a laminated fiber sheet before the application of a liquid substance is measured.
(2) A mass of the laminated fiber sheet containing the liquid substance after the application of the liquid substance is measured.
(3) A basis weight of the liquid substance is calculated from a difference between (2) and (1) above, and a proportion relative to the basis weight of the laminated fiber sheet before the application of the liquid substance in the above (1) is calculated.

In the method of using the laminated fiber sheet 10, the laminated fiber sheet 10 can be used as a variety of articles and shapes. For example, the laminated fiber sheet 10 can be used for an adhesive plaster and a bandage used by being brought into contact with a skin surface. In addition, the laminated fiber sheet 10 can be used in a tube-shaped or a glove-shaped. Specifically, examples of the tube-shaped body can include a supporter, finger sack, and the like.

In such articles, it may be preferable to have stretchability in one direction (e.g., only one direction). That is, the laminated fiber sheet 10 can be applied to the articles so as to extend and contract along the extending/contracting direction of the one direction Y in the laminated fiber sheet 10.

The terminology “stretchability in one direction” can refer to a direction in which the difference in extension percentage in two directions perpendicular to one another is equal to or greater than 20% points, and the extension percentage can be relatively large. The extension percentages in the two directions perpendicular to one another can be obtained through the measurement by applying a 1.2 N load to a sample based on the above-described (Method of Measuring Extension Percentage). The two directions perpendicular to one another can be the one direction Y and the direction X perpendicular to the one direction Y in the laminated fiber sheet 10.

### EXAMPLES

Hereinafter, embodiments of the present disclosure will be described more in detail with reference to Examples, but embodiments of the present disclosure are not limited thereto. Terms “part” and “%” in the Examples can be based on mass unless otherwise noted. The symbol “+” can mean the same value as a value in a left column. The symbol “−” below can mean that the relevant item has no value.

### Example 1

An outer sheet 2 having a median fiber diameter and a basis weight indicated in Table 1 was formed by the spunbonding method using a resin raw material PP.

A resin molten liquid was prepared using the resin raw material PP and spun by the electrospinning method. At that time, a space between a spinning nozzle and a collector was heated to 40° C. or higher by heated hot air blowing out from a peripheral area of the spinning nozzle, and spun fibers were collected on the outer sheet 2 in a state of having fluidity. In addition, a distance between the nozzle and the collector was 300 mm. As a result, an inner sheet 1 having a median fiber diameter and a basis weight indicated in Table 1 was formed, and a laminated fiber sheet sample made of the inner sheet 1 and the outer sheet 2 was obtained.

### Comparative Example 1

The outer sheet 2 prepared in Example 1 was provided alone as a fiber sheet sample of Comparative Example 1.

### Comparative Example 2

The inner sheet 1 prepared in Example 1 was provided alone as a fiber sheet sample of Comparative Example 2.

The following test was performed on the sample of each Example and each Comparative Example.

### (1) Persistent Uneven Distribution State of Liquid Substance, Wet State on Inner Side, and Dry State on Outer Side (Stuffiness Prevention) in Laminated Fiber Sheet Sample of Example 1

The following procedures (1-1) to (1-4) were executed. The results were shown in Table 2.
(1-1) From the outer sheet 2 side, 0.15 g of liquid substance (Curel Wrinkle Moisture Essence Ma, manufactured in 2022 by Kao Corporation) was applied over a 50 mm square laminated fiber sheet sample of Example 1.
(1-2) The laminated fiber sheet sample after the application was allowed to stand and recovered after a lapse of time after the application described in Table 2.
(1-3) The inner sheet 1 and the outer sheet 2 of the recovered laminated fiber sheet sample were separated to measure each mass, and from a difference from the sheet mass before the application of the liquid substance, the content mass of the liquid substance was calculated. Since the sheet mass could not be measured by separating the inner sheet 1 and the outer sheet 2 before the application of the liquid substance, values converted from the basis weights were employed.
(1-4) The ratio (mass ratio) obtained by dividing the content mass of the liquid substance in the inner sheet 1 by the sheet mass of the inner sheet 1 before the application of the liquid substance was calculated, and the mass ratio for the outer sheet 2 was calculated in the same manner. A state where the mass ratio was equal to or less than 1.5 was determined as a dry state, and a state where it was more than 1.5 was determined as a non-dry state (wet state).

### (2) Persistence of Wet State

In order to calculate mass ratios of the fiber sheet samples of Comparative Examples 1 and 2, the following procedures (2-1) to (2-5) were executed. The results were shown with the mass ratios of Example 1 in Table 3. The values of Table 2 were employed as the mass ratios of Example 1 in Table 3.
(2-1) A 50 mm square sheet was cut out, and the sheet mass before the application of the liquid substance was measured.
(2-2) From one side, 0.15 g of liquid substance (Curel Wrinkle Moisture

Essence Ma, manufactured in 2022 by Kao Corporation) was applied over the 50 mm square sheet.
(2-3) The sheet after the application was allowed to stand and recovered after a lapse of time after the application described in Table 3.
(2-4) Each mass was measured, and from a difference from the sheet mass before the application of the liquid substance, the content mass of the liquid substance was calculated.
(2-5) A ratio obtained by dividing the content mass of the liquid substance by the sheet mass before the application of the liquid substance was calculated.

### (3) Stickiness Suppression on Outer Side

In order to confirm that both the wet state on the inner side and the stickiness suppression on the outer side were achieved, the following procedures (3-1) to (3-6) were executed. The results of (3-5) and (3-6) were shown in Table 3.
(3-1) A mass of filter paper before transfer was measured.
(3-2) From one side, 0.15 g of liquid substance (Curel Wrinkle Moisture Essence Ma, manufactured in 2022 by Kao Corporation) was applied over a 50 mm square sheet.

* For the laminated fiber sheet sample of Example 1, the liquid substance was applied from the outer sheet 2 side.
(3-3) The sheet after the application was allowed to stand and recovered after a lapse of time after the application described in Table 3.
(3-4) The fiber sheet sample of each Comparative Example was placed so that a surface over which the liquid substance was applied came at an interface with the filter paper, and a load of 1.5 kg was applied from above for one minute. The laminated fiber sheet sample of Example 1 was placed so that a face on the outer sheet 2 side came at an interface with the filter paper, and a load of 1.5 kg was applied from above for one minute.
(3-5) A mass of the filter paper after the transfer was measured, and a transfer amount of the liquid substance was calculated from a mass difference between the filter paper after the transfer and the filter paper before the transfer to determine the transfer amount of the liquid substance from the outer side.
(3-6) In the above (3-1) to (3-5), for (3-4), the fiber sheet sample of each Comparative Example was placed so that a face opposite to the face over which the liquid substance was applied came at the interface with the filter paper, and the laminated fiber sheet sample of Example 1 was placed so that the face on the inner sheet 1 side came at the interface with the filter paper. Then, a load of 1.5 kg was applied, the calculation of the transfer amount of the liquid substance in (3-5) was performed to determine the transfer amount of the liquid substance from the inner side.

TABLE 1

| | | Example | Comparative | Comparative |
|---|---|---|---|---|
| | | 1 | Example 1 | Example 2 |
| Inner | Median fiber diameter | 0.5 | — | 0.5 |
| sheet 1 | P1 (μm) | | | |
| | Basis weight (g/m²) | 3 | — | 3 |
| Outer | Median fiber diameter | 20 | ← | — |
| sheet 2 | P2 (μm) | | | |
| | Basis weight (g/m²) | 17 | ← | — |

TABLE 2

| | Example 1 | | | | | |
|---|---|---|---|---|---|---|
| Time after | 10 | 20 | 30 | 40 | 50 | 60 |
| application | | | | | | |
| [min] | | | | | | |
| Mass ratio of | 1.40 | 0.84 | 0.55 | 0.65 | 0.50 | 0.69 |
| outer sheet 2 | | | | | | |
| Mass ratio of | 3.13 | 2.24 | 2.76 | 2.11 | 1.87 | 2.21 |
| inner sheet 1 | | | | | | |
| Mass ratio of | 2.24 | 2.68 | 4.99 | 3.26 | 3.72 | 3.22 |
| inner sheet | | | | | | |
| 1/Mass ratio of | | | | | | |
| outer sheet 2 | | | | | | |
| Determination | Unevenly | Unevenly | Unevenly | Unevenly | Unevenly | Unevenly |
| of uneven | distributed | distributed | distributed | distributed | distributed | distributed |
| distribution | to | to | to | to | to | to |
| | inner | inner | inner | inner | inner | inner |
| | sheet | sheet | sheet | sheet | sheet | sheet |
| Determination | Not dry | Not dry | Not dry | Not dry | Not dry | Not dry |
| of dry state | | | | | | |
| of inner sheet | | | | | | |
| 1 | | | | | | |
| Determination | Dry | Dry | Dry | Dry | Dry | Dry |
| of dry state | | | | | | |
| of outer | | | | | | |
| sheet 2 | | | | | | |

TABLE 3

| | | | Comparative | | Comparative | |
|---|---|---|---|---|---|---|
| | Example 1 | | Example 1 | | Example 2 | |
| Time after application [min] | 10 | 20 | 10 | 20 | 10 | 20 |
| Mass ratio of outer sheet 2 | 1.40 | 0.84 | 1.74 | 1.01 | 9.91 | 8.85 |
| Mass ratio of inner sheet 1 | 3.13 | 2.24 | | | | |
| Transfer amount of liquid | 0.011 | 0.003 | 0.007 | 0.000 | 0.041 | 0.021 |
| substance from outer side | | | | | | |
| Transfer amount of liquid | 0.025 | 0.019 | 0.013 | 0.003 | 0.025 | 0.018 |
| substance from inner side | | | | | | |

### Result of (1)

In Example 1, comparing the mass ratios of the inner sheet 1 and the outer sheet 2 for each of 10 to 60 minutes from Table 2, the mass ratio of the inner sheet 1 was large in any case, and it was found that the liquid substance was unevenly distributed to the inner sheet 1. In addition, the mass ratio of the inner sheet 1 exceeded 1.5, and the mass ratio of the outer sheet 2 fell below 1.5.

Therefore, as defined above, the inner sheet 1 was not dry, and the outer sheet 2 was dry. From the above, the inner sheet 1 continued to be in a non-dry state (wet state) for a long time, and the outer side continued to be in a dry state. In addition, since the fiber layer of the outer sheet 2 was able to continue to be in a dry state while the inner sheet 1 was in a wet state, the laminated fiber sheet sample of Example 1 was found to have good air permeability and excellent stuffiness prevention performance.

### Results of (2) and (3)

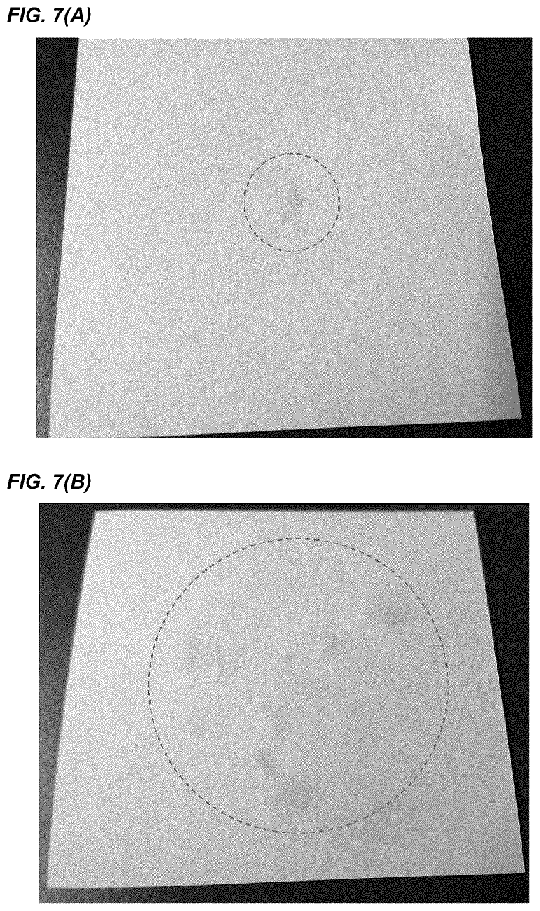

In Example 1, when the transfer amounts of the liquid substance at 10 minutes and 20 minutes were confirmed, the transfer amounts of the outer sheet 2 were less than those of the inner sheet 1 at both times. In addition, it was found that the transfer amount of the outer sheet 2 was small from FIG. 7(A) at the mass ratio of 1.4, which is determined to be dry. On the other hand, it was found that the transfer amount of the inner sheet 1 was large from FIG. 7(B) at the mass ratio of 2.24, which is determined not to be dry. As described above, it was found that, in a state where the outer sheet 2 was determined to be dry, the stickiness prevention on the outer side was provided. At the same time, in Example 1, it was found that, in contrast to the dry outer sheet 2, the inner sheet 1 adjacent thereto was in a wet state and had a large transfer amount, and therefore the action of the liquid substance on the object easily occurred. In comparison to Comparative Example 1, Example 1 was able to maintain the wet state on the inner side from the transfer amounts after 10 minutes and 20 minutes. In comparison to Comparative Example 2, Example 1 was able to suppress the stickiness on the outer side from the transfer amounts after 10 minutes and 20 minutes. Therefore, it was found that by employing the configuration of Example 1, both the stickiness suppression on the outer side and the wet state on the inner side could be achieved.

Having described embodiments and Examples according to the present disclosure, it is our intention that one or more embodiments of the present disclosure not be limited by any of the details of the description, unless otherwise specified, but rather be construed broadly within its spirit and scope as set out in the accompanying claims.

### DESCRIPTION OF SYMBOLS

1 Inner sheet
2 Outer sheet
10 Laminated fiber sheet

1. A method regarding a laminated fiber sheet wherein the laminated fiber sheet is moistened with a liquid substance, comprising:
providing the laminated sheet, wherein the laminated fiber sheet comprises:
an outer sheet; and
an inner sheet which is laminated on a face of the outer sheet, wherein, in the laminated fiber sheet,
the inner sheet and the outer sheet are insoluble to the liquid substance,
the inner sheet has a median fiber diameter of 0.3 μm or more and 5 μm or less,
the outer sheet has a median fiber diameter greater than a median fiber diameter of the inner sheet, and
the inner sheet and the outer sheet are integrated,
wherein the laminated fiber sheet is configured to develop transferability of the liquid substance from the outer sheet to the inner sheet after application of the liquid substance thereto.

2. The method according to claim 1, further comprising using the laminated fiber sheet, the using of the laminated fiber sheet including:
applying the laminated fiber sheet to an object with the inner sheet facing toward the object; and
applying the liquid substance.

3. The method according to claim 2, wherein, in a state where a face side of the inner sheet is brought into contact with the object, the liquid substance is applied from a face side of the outer sheet.

4. The method according to claim 2, wherein after the liquid substance is applied to the object and/or the laminated fiber sheet, the laminated fiber sheet is applied with the inner sheet facing toward the object.

5. The method according to claim 1,
wherein the outer sheet contains fibers having a median fiber diameter of 5 μm or more and 50 μm or less,
wherein a basis weight of the inner sheet is 1 g/m² or more and 8 g/m² or less, and/or
wherein a basis weight of the outer sheet is 8 g/m² or more and 50 g/m² or less.

6. The method according to claim 1, wherein, in the laminated fiber sheet, an extension percentage in one direction in a plane direction is 20% or more and 100% or less, and an extension percentage in a direction perpendicular to the one direction is 0% or more and 30% or less.

7. The method according to claim 1, wherein a proportion of a content basis weight of the liquid substance occupied in a basis weight of the laminated fiber sheet is 50% or more.

8. The method according to claim 2, after said applying the liquid substance, the method further includes reapplying the liquid substance from the face side of the outer sheet of the laminated fiber sheet.

9. The method according to claim 2, wherein the liquid substance is unevenly distributed to the inner sheet.

10. The method according to claim 2, further comprising putting a face side of the outer sheet in the laminated fiber sheet into a dry state after said applying the liquid substance.

11. The method according to claim 1, wherein a thickness of the inner sheet is 3 μm or more and 50 μm or less.

12. The method according to claim 2,
wherein in said applying the liquid substance, an amount of application of the liquid substance is 0.2 mg/cm² or more and 10 mg/cm² or less, and
wherein the liquid substance contains polyol that is liquid at 20° C. and, as the polyol, one kind or two or more kinds selected from ethylene glycol, propylene glycol, 1,3-butanediol, dipropylene glycol, polyethylene glycol having a mass average molecular weight of 2 000 or less, glycerin, and diglycerin.

13. The method according to claim 1, wherein a strength of fusion bonding at an interface between the inner sheet and the outer sheet that are integrated is 0.01 N/20 mm or more and 10 N/20 mm or less.

14. The method according to claim 1, wherein even when the liquid substance is contained in the laminated fiber sheet, a fiber constituting the laminated fiber sheet keeps a fiber shape.

15. A laminated fiber sheet, comprising:
an outer sheet; and
an inner sheet laminated on a face of the outer sheet, wherein
the inner sheet and the outer sheet are insoluble to a liquid substance,
the inner sheet has a median fiber diameter of 0.3 μm or more and 5 μm or less, and the outer sheet has a median fiber diameter greater than the a median fiber diameter of inner sheet,
the inner sheet and the outer sheet are integrated, and
the laminated fiber sheet is configured to contain the liquid substance such that the liquid substance is unevenly distributed to the inner sheet.

16. The laminated fiber sheet according to claim 15, wherein the laminated fiber sheet is configured such that a face side of the outer sheet is put into a dry state by the uneven distribution of the liquid substance.

17. The laminated fiber sheet according to claim 15,
wherein the outer sheet contains fibers having a median fiber diameter of 5 μm or more and 50 μm or less,
wherein a basis weight of the inner sheet is 1 g/m² or more and 8 g/m² or less, and/or
wherein a basis weight of the outer sheet is 8 g/m² or more and 50 g/m² or less.

18. The laminated fiber sheet according to claim 15, wherein the inner sheet and the outer sheet are adjacent and integrated by fusion bonding of fibers constituting the inner sheet and fibers constituting the outer sheet.

19. The laminated fiber sheet according to claim 15, wherein the inner sheet and the outer sheet contain a material of a same family, and the material of the same family is selected from an olefin-based resin, a diene-based resin, a urethane-based resin, and/or a copolymer thereof.

20. The laminated fiber sheet according to claim 15, wherein, in the laminated fiber sheet, an extension percentage in one direction in a plane direction is 20% or more and 100% or less, and an extension percentage in a direction perpendicular to the one direction is 0% or more and 30% or less.
